(19) United States
(12) Patent Application Publication
(10) Pub. No.: US 2025/0213351 A1
(43) Pub. Date: Jul. 3, 2025

(54) ZONAL ABERRATION OPTIMIZED CONTINUOUS FOCUS LENS

(71) Applicant: Krishnakumar Venkateswaran, Aliso Viejo, CA (US)

(72) Inventor: Krishnakumar Venkateswaran, Aliso Viejo, CA (US)

(21) Appl. No.: 19/005,636

(22) Filed: Dec. 30, 2024

Related U.S. Application Data
(60) Provisional application No. 63/616,352, filed on Dec. 29, 2023.

(51) Int. Cl. A61F 2/16

(52) U.S. Cl. CPC A61F 2/1645; A61F 2/1618; A61F 2/1654

(57) ABSTRACT

An intraocular lens including an optic having an anterior surface and a posterior surface disposed about an optical axis is provided. At least one of the anterior surface and the posterior surface has a toric shape and at least one of the anterior surface and the posterior surface including a combination of zones in its surface with alternating signs for a wavefront aberration component defined using annular terms for balancing focus to maintain modulation transfer function (MTF) values at different spatial frequencies. The intraocular lens may be monofocal or may be multifocal, depending on the specific implementation.

### CROSS-REFERENCE TO RELATED APPLICATIONS

The present application claims priority from U.S. Provisional Patent Application No. 63/616,352, filed on Dec. 29, 2024, the disclosure of which is hereby incorporated by reference in its entirety.

### TECHNICAL FIELD

This disclosure relates to intraocular lenses (IOLs), and more particularly to zonal aberration optimized continuous focus lenses.

### BACKGROUND

IOLs including an optic with either none, one, or more haptics for positioning the optic within an eye are known. Haptics are generally appendages that hold the optic in place after it is implanted in the eye, to correct refractive errors. Multifocal lenses represent one type of IOL providing a range of vision including distance vision, intermediate vision and/or near vision.

A class of lenses which can provide more than one focal plane is referred to as refractive multifocal. In refractive multifocal lenses, an optic is divided into multiple refractive zones, and light from a particular zone is directed to only one of the foci using only refractive power. The zones can be concentric about the optical center or non-axis-symmetric. Refractive multifocal lenses form two or more foci at different focal distances to provide far, intermediate, and/or near vision.

Diffractive multifocal lenses are another class of lenses which can provide more than one focal plane. Diffractive lenses leverage the concept of diffraction. In particular, diffractive steps cause light to be out of phase with light transmitted through adjacent zones (i.e., there is a phase delay between adjacent zones). The radial boundaries that separate the zones are chosen to achieve particular optical powers in diffractive lenses, whereas the radius of the surfaces achieve particular optical powers in refractive lenses.

A well-known example of a figure of merit for measuring the performance of visual systems is known as a Modulation Transfer Function (commonly referred to as an “MTF”). An MTF of an optical system is a measure of the proportion of contrast of an input object that the optical system is able to maintain when an image of the object is produced. An MTF can be measured as a function of spatial frequency (e.g., line pairs per mm at the retina). Generally, the MTF values for a given optical system decrease with an increase in the spatial frequency.

Efficiency in delivering images at multiple focal planes is observed through modeling and measuring MTF. Techniques for extending the depths of focus of monofocal IOLs (i.e., without multiple peaks in the MTF curve) to obtain distance vision as well as nearer vision have been proposed. IOL techniques to provide an extended depth of focus (EDOF) include a) providing an IOL with a central refractive zone with add power; b) providing an IOL with high magnitude positive or negative spherical aberration ac; and c) providing an underlying refractive IOL with a relatively low-power add diffractive profile (diffractive add of 1.5 Diopter or less). Each such extended depth of focus technique has provided limited improvement to wearers' vision quality.

Conventionally, in refractive design techniques, the phase delay at a symmetrical or asymmetrical zone provides focus at the add distance for which the zone is designed. These designs tend to reduce the bifocality of the lens by decreasing the percentage of light sent to the near focus in favor of light sent to the far focus, since wearers of multifocal lenses tend to prefer peak vision performance for distance vision. Such lenses suffer from similar drawbacks as the more bifocal lenses with regard to presence of multiple peaks in MTF. Conventionally, in diffractive lenses a low-power add profile is selected such that the phase delay between adjacent zones is a fraction of the wavelengths of a design wavelength (usually around 550 nm for visible light). These lenses tend to provide multifocality, such that light is divided between a central focus corresponding to a zeroth order of the diffractive profile, and a near and a far focus corresponding to a +1 order and a −1 order of the diffractive profile. Such lens configurations tend to cause multiple peaks in the MTF with varying light levels depending on the characteristics of the diffractive steps. These designs tend to direct light symmetrically about a central focus to each of the near and far foci, resulting in loss of light energy, resulting in a peak at far vision, and lens performance may be compromised.

Irrespective of the refractive or diffractive design, for a given spatial frequency, each focus of a IOL (i.e., near, intermediate, or far focus) manifests itself in a through-focus MTF plot as a peak in MTF values, with lower MTF in between the focal planes. For an individual wearer of an IOL, a region of lower MTF values may be large enough to permit vision depending on the broadening and flattening of MTF peaks that occurs for the individual due the ocular aberrations of the individual's eye and his/her pupillary response.

Multifocal lenses are known to provide a beneficial increase in the range of vision. However, a significant proportion of patients with lenses employing multifocal techniques have been known to suffer visual confusion. Therefore, there is a need for alternatives for extending the depth of focus of ophthalmic lenses, without multiple peaks in the MTF curves of resultant lenses, and with more efficient use of light energy.

Multifocal lenses have inherent optical aberrations such as spherical aberration associated with their design. Hence the optical performance of these lenses is highly dependent on the optical elements present in the human eye (e.g., optical characteristics of the cornea). In designing IOLs and lenses generally, optical performance can be determined by measurements using “model eye” or by calculations, such as ray tracing. However, all these lens designs suffer from lower MTF and hence lower image contrast as the corneal aberration profile changes. Accordingly, there is still a need for IOLs that can provide pseudo-accommodative optical power while providing sharp optical images over a wide range of aberrations of the cornea, as well as over different pupil sizes.

It is with respect to these and other considerations that embodiments have been described in the following disclosure.

### SUMMARY

Embodiments herein generally relate to intraocular lens designs with an insensitivity to corneal aberrations, such as spherical aberrations. Namely, certain embodiments relate to continuous aberration compensation intraocular lenses. For example, embodiments described or otherwise contemplated substantially provide IOLs with pseudo-accommodative optical power that provide sharp optical images over a wide range of aberrations of the cornea and pupil sizes.

One embodiment relates to a monofocal intraocular lens, including an optic having an anterior surface and a posterior surface disposed about an optical axis. At least one of the anterior surface and the posterior surface has a toric shape and at least one of the anterior surface and the posterior surface including a combination of zones in the surface with results having alternating signs for a wavefront aberration component defined using annular terms for balancing focus and maintaining MTF at different spatial frequencies.

According to aspects of the present disclosure, in order to provide extension in depth of focus and reduce the likelihood of photic phenomena, a monofocal IOL can be used having a best focus for far vision and a depth of focus extending toward a nearer, intermediate range. In such a monofocal IOL, the MTF maintains values across spatial frequencies important for high contrast distance and intermediate vision, and at the same time is relatively independent of the aberrations of the other optical elements, such as the cornea. The through-focus MTF curve of such a “monofocal IOL with extended depth of focus” IOL is designed such that there is a single peak (i.e., a single local maximum) corresponding to far vision (also corresponding to the absolute maximum) and, for positive add powers from the maximum single peak (i.e., the myopic side of best focus), with the MTF>0.15 at 50 lp/mm and >0.19 at 100 lp/mm such that there is a decreasing yet visually-useful level of MTF extending in the myopic direction from the peak. In such embodiments, the MTF is non-increasing until the first zero in MTF is reached. In some embodiments, it is beneficial to the goal of reducing photic phenomenon caused due to images from multiple focal planes. Hence the MTF is monotonically decreasing until a first zero in the MTF is reached.

To achieve such performance, embodiments of the present disclosure may include a refractive lens with multiple zones on the surface with the minimum of one refractive power and maximum of the next refractive power merged, such that the aspheric component minimally impacts the visual performance of the IOL at any radii. The refractive profile of the refractive lens includes a combination of two or more refractive profiles, each having a power so as to maintain the MTF values without void in vision.

Another aspect of the present disclosure may be directed to a set of intraocular lenses, including refractive zones. Each lens is configured according to aspects or embodiments set forth above. Each of the lenses of the set has a different refractive, base dioptric power than one another. To facilitate the spreading of the energy along the depth of focus, the zone profiles may be selected to have a phase delay, relative to aqueous fluid with any range of power designed at 546 nm light, which in conjunction with the overlap method of combining the profiles tends to spread the light along the depth of focus without affecting the aspheric component over the full optic. The spread in light at the focal plane is highly dependent on the aberration compensation of the cornea. In some embodiments, the plurality of lenses of the proposed design each have a range of radii with an aberration component individually optimized for each radius, resulting in optimized aberration for the entire optic resulting in less sensitivity to a range of human corneal aberrations.

In a still further aspect, an intraocular lens includes an optic having an anterior surface and a posterior surface disposed about an optical axis, at least one of the anterior surface and the posterior surface having a toric shape and a shaped surface selected from the anterior surface and the posterior surface. The shaped surface includes a plurality of zones positioned concentrically. Adjacent ones of the plurality of zones having alternating signs for a wavefront aberration component defined using annular terms for balancing focus, the plurality of zones being selecting to maintain modulation transfer function (MTF) values providing functional vision over a range of focal distances.

Although the above examples refer to refractive lens designs, it is understood that similar features may be implemented in a diffractive lens arrangement, in further example aspects.

In a yet further aspect, the present disclosure provides an ophthalmic lens, such as an intraocular lens, that includes an optic having an anterior surface and a posterior surface about an optical axis. At least one of the surfaces (e.g., the posterior surface) has a profile characterized by superposition of a base profile and one or more auxiliary aspheric profile or auxiliary non-aspheric profiles. The auxiliary profiles are characterized within boundaries and serves as a transition region, where an optical path difference across the transition region (i.e., the optical path difference between the inner and the outer radial boundaries of the transition region) corresponds to any arbitrary radius (e.g., to) for a design wavelength (e.g., a wavelength of about 550 nm) and can include an aspheric component. The transition region of these auxiliary profiles can extend from a defined inner radial boundary to an outer radial boundary. In many embodiments, the transition region can be adapted to provide a monotonic change in optical path difference relative to its inner radial boundary as a function of MTF at a specified spatial frequency to provide visual performance. A monotonic change in the optical path difference can be characterized by a continuous increase or decrease as a function of radial distance of the changes in radius of the surface. By way of example, the change in surface radius can be characterized by a linear or a non-linear change or a succession of radius changes with aspheric component optimized for each zone and the combination of zones to provide optimal aberrations for the entire range of pupil sizes covering photopic to mesopic light levels.

The term “anterior”, when used herein with reference to an intraocular lens, refers to a feature on the lens tending toward the direction of the cornea of an eye in which the lens to be implanted, and term “posterior” refers to a feature on the lens tending toward on the retina of the eye.

A wavelength of light specified in nanometers (nm) herein refers to the wavelength when said light is propagated in vacuum. For example, 546 nm light refers to light having a wavelength of 546 nm when propagating in a vacuum.

The term “monofocal” as used herein refers to a lens having a single peak (i.e., a single local maximum; and the single peak also being the absolute maximum) in the MTF before a first zero in the MTF is reached for positive add powers.

The term “first zero” is defined herein to mean a first local minimum after the absolute maximum (i.e., best focus) in a through-focus MTF plot.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIG. 1 illustrates a schematic illustration of a human eye after a natural lens has been removed and an intraocular lens has been surgically implanted in the capsular bag of the eye.

FIG. 2 illustrates a schematic side view of an IOL to illustrate a surface profile and/or optical power, according to an embodiment.

FIG. 3 illustrates a schematic side view of an IOL to illustrate a surface profile and/or optical power, according to an embodiment.

FIG. 4 illustrates a lens design in which the inner regions of an IOL have a longer focal length than the outer regions facilitating vision at myopic distances and these regions are alternating the location of focus and optimized aberration pattern resulting in a through focus MTF profile, according to an embodiment.

FIG. 5 illustrates graphs of a modulation transfer function at 50 lp/mm of the lens modeled using different corneal spherical aberrations of 3 mm and 4.5 mm respectively, according to an embodiment.

FIG. 6 illustrates results of optical measurements with a 3 mm aperture and 0.0 spherical aberration cornea under the same lighting conditions, according to an embodiment.

FIG. 7 illustrates a through focus curve comparison illustrating performance of a lens developed in accordance with examples of the present disclosure.

While various embodiments are amenable to various modifications and alternative forms, specifics thereof have been shown by way of example in the drawings and will be described in detail. It should be understood, however, that the intention is not to limit the claimed subject matter to particular embodiments described. On the contrary, the intention is to cover all modifications, equivalents, and alternatives falling within the spirit and scope of the subject matter as defined by the claims.

### DETAILED DESCRIPTION

Various embodiments will be described in detail with reference to the drawings. Reference to various embodiments does not limit the scope of the claims attached hereto. Additionally, any examples set forth in this specification are not intended to be limiting and merely set forth some of the many possible embodiments for the appended claims.

As briefly described above, embodiments of the present disclosure are directed to IOLs with pseudo-accommodative optical power. In general, the disclosed apparatus can provide sharp optical images over a wide range of aberrations of the cornea over various pupil sizes.

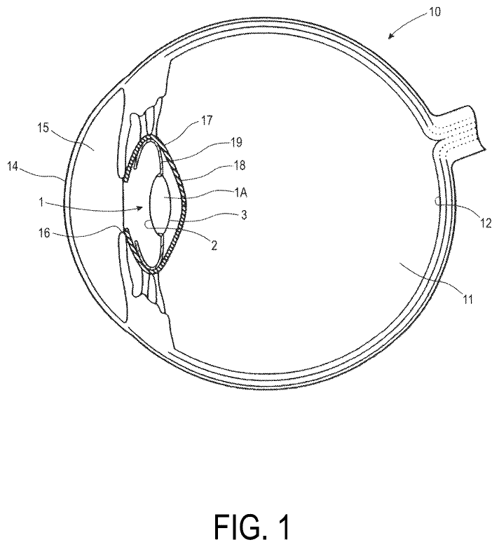

For reference, FIG. 1 can be understood to depict the general arrangement of a human eye 10 after an intraocular lens 1 has been surgically implanted. Light enters from the left of FIG. 1, and passes through cornea 14, anterior chamber 15, iris 16, and enters capsular bag 17. Capsular bag 17 houses intraocular lens 1 in addition to aqueous fluid which occupies the remaining volume and equalizes the pressure in the eye 10. After passing through intraocular lens 1, light exits posterior wall 18 of the capsular bag 17, passes through posterior chamber 11, and strikes retina 12. The retina detects the light and converts it to a signal transmitted through an optic nerve to the brain.

Intraocular lens 1 has an optic 1A that has a refractive index greater than the aqueous fluid that surrounds it and, typically, the refractive power of an intraocular lens is in the range of about 5 Diopters to about 30 Diopters to compensate for the loss of natural lens which it typically replaces.

Optic 1A has an anterior surface 2 facing away from the retina 12 and a posterior surface 3 facing toward the retina 12. As illustrated, an optic 1A is held in place by one or more haptics 19, which couple optic 1A to the capsular bag 17. The one or more haptics may be of any known or yet to be developed configuration (e.g., plate, wire, C-loop, J-loop), and may be of an accommodating or non-accommodating type. In some embodiments, the IOL may have no haptics.

Optic 1A intraocular lens 1 may be disposed adjacent to, and even pressed against, the posterior wall 18, for example, to reduce cellular growth on optic 1A. Alternatively, optie 1A may be positioned within the capsular bag 17 in a position spaced away from the posterior wall 18, for example, to allow accommodative movement of optic 1A of the intraocular lens 1 along the optical axis.

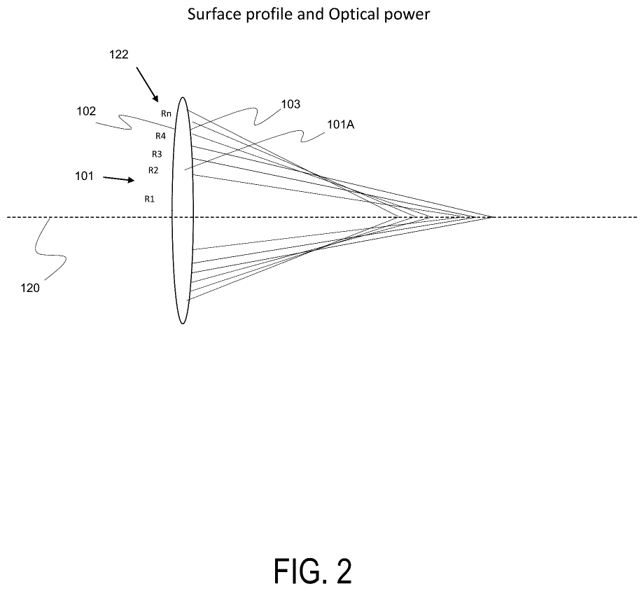

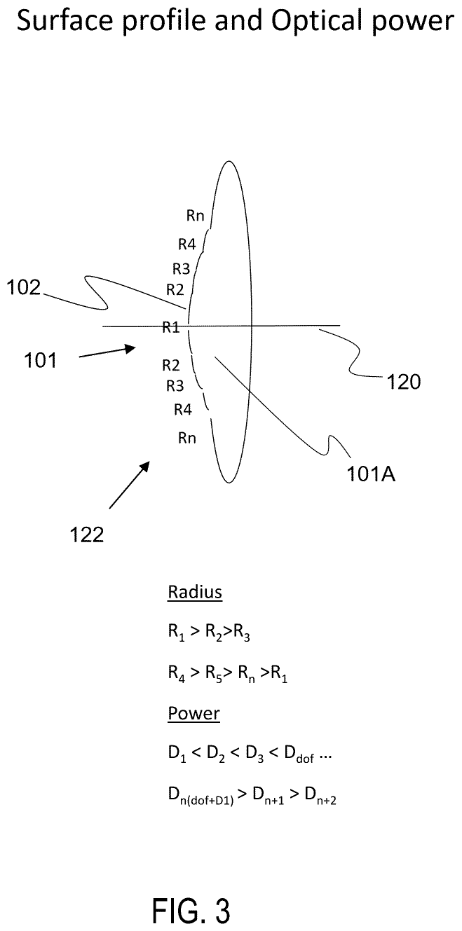

Various embodiments of the present disclosure are understood based on FIGS. 2-6 and related disclosure. In general, the radius of the anterior and posterior surfaces of a lens result in a combination of optical powers over the desired pupil aperture such that 75% of area occupied by 0.5D from base power to myopic power and 25% of the area occupied by 0.5D from mid-point of power from base power to 0.5D myopia. This overlap in refractive power displaced in a radial direction provides a through focus MTF curve with a flat top, resulting in insensitivity to optical power over a range of 0.5D from base power.

In some embodiments, the present disclosure provides an ophthalmic lens 101 (e.g., an IOL) that includes an optic 101A having an anterior surface 102 and a posterior surface 103 described about an optical axis 120. At least one of the surfaces (e.g., the anterior surface 102) has a profile characterized by superposition of aspheric profiles. The auxiliary profile can include two or more regions (or zones) 122, where an optical path difference across the transition region corresponds to a desired level of MTF over the different range of depth of focus. The transition region of these profile can extend between two zone boundaries depending on the MTF value to be provided at that depth of focus.

The profile Z_{Total} of the surface formed as superposition of a base profile and a series of aspheric or non-aspheric profiles defined by the following relation:

$${Z}_{Total} = {Z}_{base} + {Z}_{1} + {Z}_{2} + {Z}_{3} + … … + {Z}_{n}$$

The number of zones is such that the width of zone is at least a few tens of wavelengths larger than the design wavelength so as not to induce any diffractive effects. In general, any profile is described using the equation:

$${Z}_{Profile} \{ {cr}^{2} / [ 1 + sqrt ( 1 - ( 1 + k ) {c}^{2} {r}^{2} ] ] + ( {a}_{2} {r}^{2} + {a}_{4} {r}^{4} + {a}_{6} {r}^{6} + … )$$

In the above, r denotes a radial distance from the optical axis, c denotes a base curvature of the surface, k denotes a conic constant, a₂ is a second order deformation constant, a₄ is a fourth order deformation constant, a₆ is a sixth order deformation constant.

Each zone has such a profile such that the there is an overlap in power of the zones such that the total power of the last two or more zones within the aperture is equal to the total power of the zones at the central two or more zones. This power overlap also results in frequencies including 100 lp/mm have MTF value >=0.2 providing functional vision in cataract patients implanted with such a lens. These zones have their aberrations described using a set of orthogonal polynomials, such as annular Zernike polynomials. As an illustration, combining the defocus term (Z_{annularFocus}) and spherical aberration term (Z_{annularSA}), the phase component can be described using the following equation:

$${Z}_{annular} = [ sqrt ( 3 ) ( - 2 {r}^{∧} 2 + {e}^{∧} 2 + 1 ) / ( {e}^{∧} 2 - 1 ) ] + [ Sqrt ( 5 ) ( 6 {r}^{∧} 4 - 6 ( {e}^{∧} 2 + 1 ) {r}^{∧} 2 + {e}^{∧} 4 + 4 {e}^{∧} 2 + 1 / {( {e}^{∧} 2 - 1 )}^{∧} 2 ]$$

In this equation, e is the obscuration ratio and r the normalized radius. For a multi component surface, the phase surface can be described using the following equation:

$${Z}_{Total} = [ sqrt ( 3 ) ( - 2 {{r}_{1}}^{∧} 2 + {{e}_{1}}^{∧} 2 + 1 ) / ( {{e}_{1}}^{∧} 2 - 1 ) ] + [ sqrt ( 5 ) ( 6 {{r}_{1}}^{∧} 4 - 6 ( {{e}_{1}}^{∧} 2 + 1 ) {{r}_{1}}^{∧} 2 + {{e}_{1}}^{∧} 4 + 4 {{e}_{1}}^{∧} 2 + 1 / {( {{e}_{1}}^{∧} 2 - 1 )}^{∧} 2 ] + [ sqrt ( 3 ) ( - 2 {{r}_{2}}^{∧} 2 + {{e}_{2}}^{∧} 2 + 1 ) / ( {{e}_{2}}^{∧} 2 - 1 ) ] + [ sqrt ( 5 ) ( 6 {{r}_{2}}^{∧} 4 - 6 ( {{e}_{2}}^{∧} 2 + 1 ) {{r}_{2}}^{∧} 2 + {{e}_{2}}^{∧} 4 + 4 {{e}_{2}}^{∧} 2 + 1 / {( {{e}_{2}}^{∧} 2 - 1 )}^{∧} 2 ] + … + [ sqrt ( 3 ) ( - 2 {{r}_{n}}^{∧} 2 + {{e}_{n}}^{∧} 2 + 1 ) / ( {{e}_{n}}^{∧} 2 - 1 ) ] + [ sqrt ( 5 ) ( 6 {{r}_{n}}^{∧} 4 - 6 ( {{e}_{n}}^{∧} 2 + 1 ) {{r}_{n}}^{∧} 2 + {{e}_{n}}^{∧} 4 + 4 {{e}_{n}}^{∧} 2 + 1 / {( {{e}_{n}}^{∧} 2 - 1 )}^{∧} 2 ]$$

In the preceding equation, rₙ−Iₙ₋₁, which refers to the aberration within the annulus, is optimized to the full optic on the anterior or posterior side as per design. Additionally, A1, A2, A3 can be selected to alternate the power profile, such as that the MTF can be added in phase at the focal plane as required.

The radius over which the annular Zernikes are described using the mid-point of the optical power of the depth of focus from the base power it is designed for. As an illustration, if the lens has a base power of 20.0D and designed for a 1.5D depth of focus extension over a 1.5 mm pupil size, then the radius of the zones on the optical surfaces are described by −pD²+pD+DoF/2, where p is the radius of the individual aspheric zone, D is the optical power of that zone and DoF is the depth of focus for which the lens is designed.

The base power is extended out to a myopic power of xD, and then the rest of the regions is extended from xD to base power—yD, where yD<base power. This combination of xD and yD determines the depth of focus of the lens. The aberration component over these regions can be defined using a complete set of orthogonal polynomials such as the annular Zernike polynomials.

Accordingly, an embodiment of the disclosure includes a monofocal intraocular lens, comprising: an optic having an anterior surface and a posterior surface disposed about an optical axis, where at least one of the anterior surface of the posterior surface is toric. Additionally, at least one of said anterior or posterior surfaces includes a combination of such zone results in a surface with alternating signs for wavefront aberration component defined using annular terms for balancing the focus, so as to maintain the MTF at different spatial frequencies.

In some examples, such a monofocal intraocular lens has an MTF at 50 lp/mm and 100 lp/mm>=0.50 and >=0.3 MTF units respectively, resulting in superior image quality at different myopic focal planes.

In some further examples, such a monofocal intraocular lens may have different area ratios of the annular zones, resulting in a family of lenses with a continuous range of focus with very low blur levels.

In some further examples, such a monofocal intraocular lens may have aberration in different zones is such that the through focus MTF at different frequencies stay higher than 0.3MTF units, providing functional vision at depth of focus >1.0D across corneas of all aspheric values from 0.0 μm to 0.3 μm.

In some further examples, such a monofocal intraocular lens may include superposition of annular zones which are power dependent, such that the number of annular regions is split into base region and myopic region. In such examples the base region differs from myopic region by the depth of focus for which the lens is designed. As an illustration, if the base power is 20D, then the depth of focus is set to 0.5D and the myopic region starts at 21D separated, extending the depth of focus by 0.75D in the subsequent annular regions.

In example implementations, higher order aberrations are optimized to any intended corneal spherical aberrations, such that the depth of focus is consistent for at least 0.25 μm on both positive and negative side of the spherical component of the cornea for which the lens is designed.

In example implementations, the spherical component of each annular zone is optimized for the mid-range cornea. In particular, the spherical aberration is balanced for 4.5 mm aperture such that the MTF at 50 lp/mm stays above 0.30 MTF units across any human cornea with the spherical aberration in the range −0.1 μm and 0.30 μm, including all ISO corneas.

In example implementations, the regions of alternating power can extend up to any predefined radius or to the physical extent of the lens itself to enable depth of focus over a range of pupil sizes.

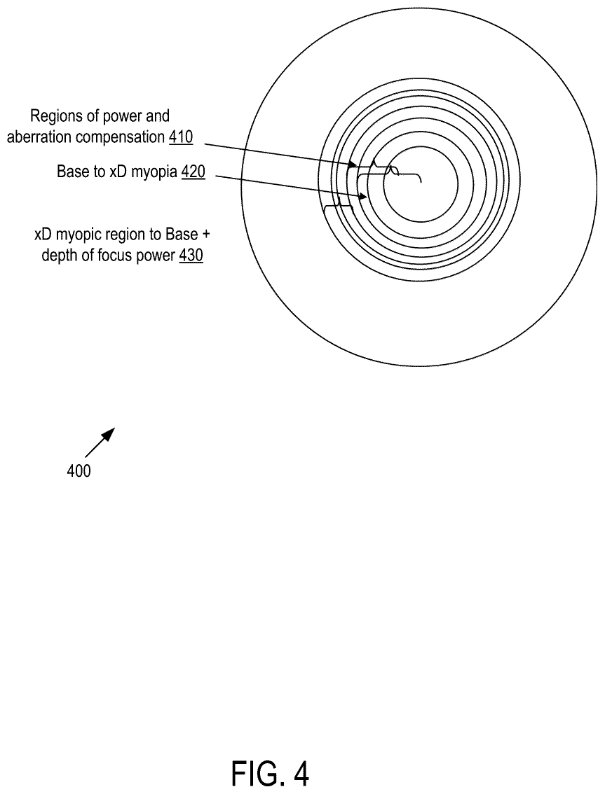

In FIG. 4, a diagram 400 illustrates regions of power and aberration compensation can be seen at 410 and base to xD myopia can be seen labeled at 420. Finally, xD myopic region to Base+depth of focus power is indicated at 430.

As shown in FIG. 4, embodiments of the present disclosure have aberration regions defined using polynomials optimized for the optic on the anterior or posterior side, and can be defined as annular Zernike polynomials with the obscuration ratio defined by the radius of each region.

The cross section of such a lens is such that the power profiles of the adjacent regions are continuous over xD and overlap with the next set of zones, such that the MTF at every focal plane is continuous and is maintained at values providing functional vision over a range of foci.

Embodiments of the present disclosure have aberration regions defined either empirically or analytically for the optics on the anterior or posterior side, with the myopic region either added or subtracted from the base power to provide a range of focus.

Embodiments of the present disclosure include lens designs that are insensitive to corneal aberrations. That is, the optimized zones provide compensation for corneal aberrations over the designed pupil size irrespective of the phase introduced in the wavefront entering the lens.

In the figures, an illustration of an embodiment of the present disclosure is shown in which the inner regions have a longer focal length than the outer regions facilitating vision at myopic distances and these regions are alternating the location of focus and optimized aberration pattern resulting in a through focus MTF profile which is relatively insensitive to the aberration profile of an incoming wavefront.

The cross section of the lens in the figures can be understood as a lens with a power profile as described in the figures.

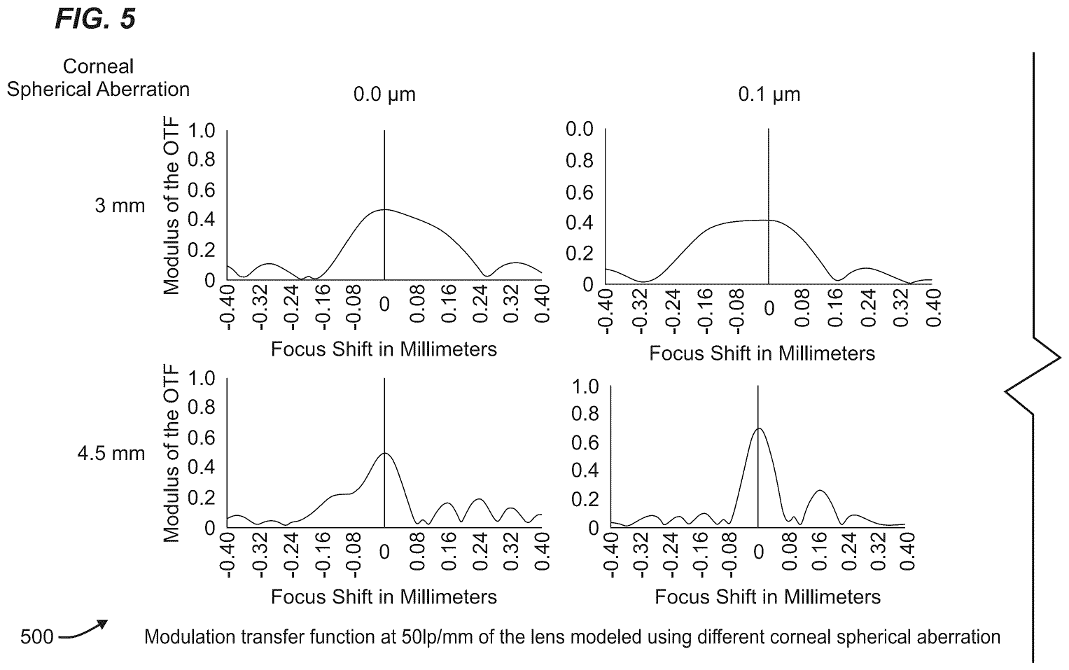

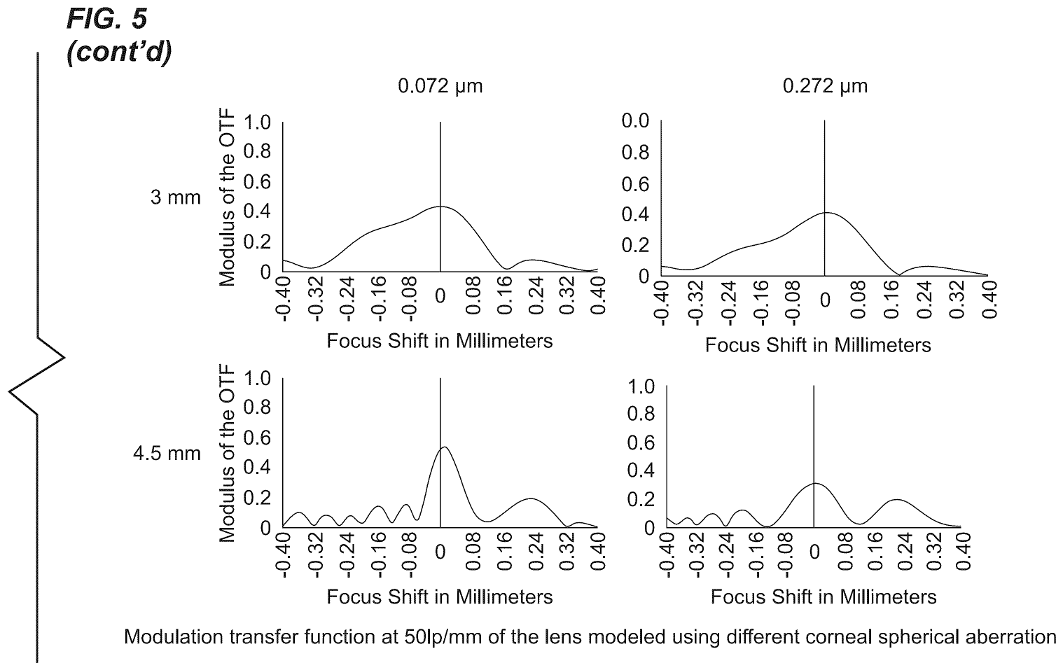

FIG. 5 illustrates a series of plots 500, showing representative MTF curves in the various plots. A modulation transfer function at 50 lp/mm of the lens modeled using different corneal spherical aberrations. Corneal spherical aberrations of 3 mm and 4.5 mm are used. The x-axis of the respective charts indicating the focus shift in millimeters and the y-axis representing the Modulus of the OTF.

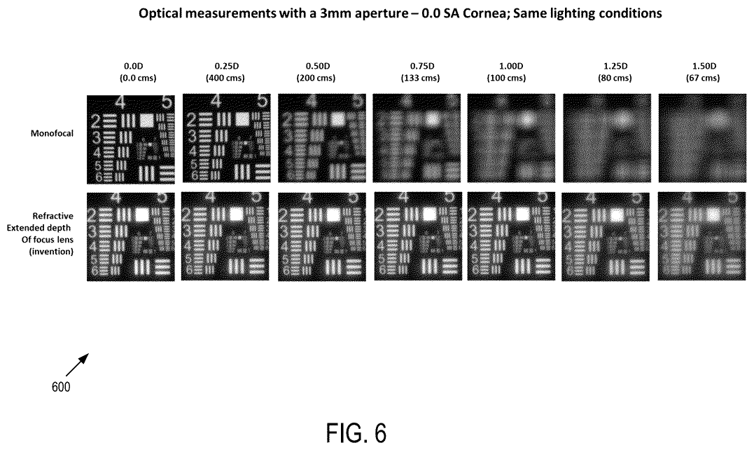

FIG. 6 illustrates a graph 600 illustrating results of optical measurements with a 3 mm aperture and 0.0 spherical aberration cornea under the same lighting conditions, according to an embodiment. The clarity of the refractive extended depth of focus lens can be seen across a wide range of focal lengths, in contrast to monofocal optical results can be seen. Accordingly, the benefits of a continuous aberration compensation intraocular lens, in terms of clarity across a wide range of focal distances, can be understood pursuant to this disclosure.

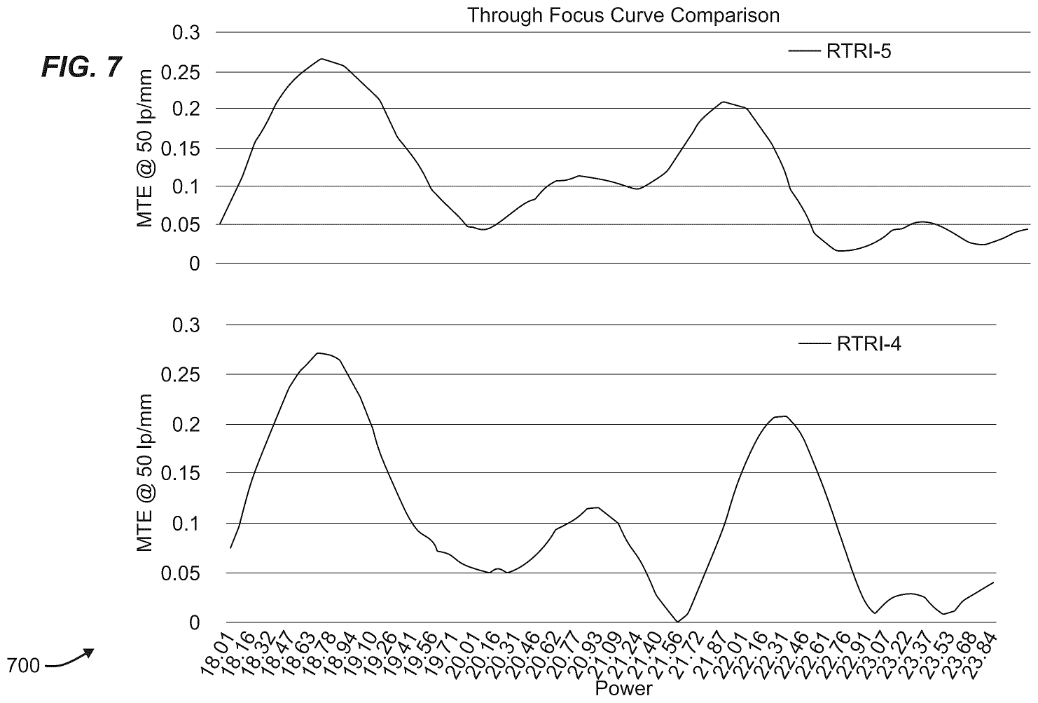

FIG. 7 illustrates a comparative line graph 700 showing optical power measurements and MTF for a lens according to the present disclosure, as compared to a preexisting lens. As illustrated in this example, certain features of the monofocal optical lenses described above may be extended to a bifocal or multifocal lens as well, in accordance with the present disclosure. In the present application, an optical lens having spherical aberrations selected and sized to extend far-distance optical power across a wider range enables improved vision over a variety of focal lengths. By contrast, the arrangement seen in the lower through focus curve has a sharp dropoff surrounding the far range focal distance.

In particular, the lens described by the top graph represents a refractive trifocal IOL with a unique through-focus curve pattern that is designed to manipulate energy delivery at different focal points through zone radius control. The lens as described provides distance, intermediate, and near vision capabilities, and maintains performance across different corneal profiles. Furthermore, an energy distribution accomplished via the lens can be controlled through appropriate zone sizing.

In general, the lens described by the top graph uses multiple zones with different radii and powers. Additionally, a requirement that R1>R2>R3 that was present in other lens designs, including those outlined above, need not be strictly followed, allowing more design flexibility. To achieve the lens design as described, a zone sizing process is performed by calculating area formulas to determine a desired power at various zones/focal lengths; πr² is used for the innermost zone, and π(R2²−R1²) for subsequent zones. Using a percentage-based energy distribution, power is manipulated to deliver 40% to distance, and 20% from other zones, across a 1.5 mm radius pupil zone. In this instance, zones are split into six different region with varying powers; however, in alternative embodiments, more or fewer zones may be included.

In example implementations, a design process to develop an IOL in accordance with this embodiment involves starting with a design having a predetermined power level (e.g., 10 diopter power, requiring a −53 mm radius). A conic constant is optimized for an initial (central) zone and maintained constant across other zones. A surface design on either an anterior or posterior side of the lens may be selected. The selection

In accordance with the present disclosure, a refractive trifocal intraocular lens can be manufactured using the following parameters (again, using a 10 D optical power example):

TABLE 1
Example 10 D Intraocular Refractive Lens Dimensions

| | | | | | Spherical | Add | | | | | |
|---|---|---|---|---|---|---|---|---|---|---|---|
| | | | | | Eq | power | | | | | |
| | Norm | Zone | SEQ | CYL | Power | @ | | | Anterior | Anterior | Central |
| | Radius | (mm) | (D) | (D) | (D) | 1.15 mm | Power | Target | Radius | Conic | Thickness |
| Base | 0.33 | 0.50 | 10.0 | 0.0 | 10.0 | 1.0 | 10.0 | 10.0 | 21.0 | −17.872 | 0.515 |
| EDoF | | | | | | | | | | | |
| | 0.50 | 0.750 | | | | | 8.0 | | | | |
| | 0.67 | 1.0 | | | | | 9.0 | | | | |
| | 0.8 | 1.2 | | | | | 11.5 | | | | |
| | 0.87 | 1.3 | | | | | 12.0 | | | | |
| | 1.0 | 1.5 | | | | | 12.0 | | | | |

A further example is provided in Table 2, below, representing a 7 D optical power example:

TABLE 2
Example 7 D Intraocular Refractive Lens Dimensions

| | | | | | Spherical | Add | | | | | |
|---|---|---|---|---|---|---|---|---|---|---|---|
| | | | | | Eq | power | | | | | |
| | Norm | Zone | SEQ | CYL | Power | @ | | | Anterior | Anterior | Central |
| | Radius | (mm) | (D) | (D) | (D) | 1.15 mm | Power | Target | Radius | Conic | Thickness |
| Base | 0.42 | 0.63 | 7.0 | 0.0 | 7.0 | | 7.0 | 7.0 | 39.7401 | −150.385 | 0.389 |
| EDoF | | | | | | | | | | | |
| | 0.59 | 0.89 | | | | | 5.0 | | | | |
| | 0.73 | 1.09 | | | | | 6.0 | | | | |
| | 0.84 | 1.259 | | | | | 8.5 | | | | |
| | 0.94 | 1.408 | | | | | 9.0 | | | | |
| | 1.0 | 1.5 | | | | | 9.0 | | | | |

It is noted that lenses developed in accordance with the present disclosure are useable independently of corneal specifications, unlike existing commercial lenses. Additionally, performance of such lenses improves as corneal spherical aberration increases, and optimal performance is maintained across different corneal aberration profiles.

Example implementations of the present disclosure enable a design in which intermediate vision location may be manipulated between far and near focal points by manipulating the power levels delivered in different focal zones. Phase addition principles are used to control focal points between distance and near vision, and may be done systematically.

In accordance with the present disclosure, although specific surface profiles are discussed and enabled, it is noted that surface changes in the intraocular lens are generally seamless and difficult to identify visually. Additionally, the surface profile of even a multifocal lens created in accordance with the arrangement shown in FIG. 7 have a surface profile that appears similar to a conventional monofocal lens arrangement.

Additional lenses may be created in accordance with the present disclosure in a range of power levels from 7.0 D to 17 D. Specific zones and radii are designed proportionally to maintain power levels across a variety of focal distances in accordance with the above examples at 7.0 D and 10 D.

Referring to the principles described herein, including the features described above in conjunction with FIGS. 1-7, a number of features and advantages are presented herein. For example, a zone design methodology is provided in which specific calculations are used for zone sizing using πr² for internal zones and π(R2²-R1²) for subsequent zones concentrically outwardly. Additionally, energy distributions are selected such that approximately 40% is allocated to distance vision, and 20% is allocated to subsequent inward zones. Still further, in some examples, either a sequential radius relationship or a non-sequential radius relationship is selected in which a traditional R1>R2>R3 progression may or may not be followed.

In addition, in accordance with the present disclosure, power distribution may be controlled such that specific area ratios are provided in which 75% area is used for base power to myopic power and 25% area is used from mid-point power to 0.5D myopia. Additionally, an overlap method is used between adjacent zones for maintaining continuous MTF values. Intermediate vision locations are manipulated between distance and near focal points.

Still further, the present disclosure contemplates design of intraocular lenses that are independent of corneal geometry across a range of corneal profiles. For example, corneal profiles (MTF maintenance above 0.30 units across-0.1 to 0.30 micrometer spherical aberration range) are contemplated and may be accommodated, as compared to existing commercial lenses that are limited to specific corneal types and therefore require separate lenses for each of a large number of corneal types.

Such features are implemented in accordance with an optimization process in which first, a central zone is defined with base power. For example, an initial 10 diopter power may require a −53 mm radius. Zone boundaries are defined extending to 1.5 mm radius. Power delivered in the central zone is calculated using the area of the central zone, and subsequent zone areas are calculated to enable power distribution across zones, with approximately 40% energy delivered to distance vision, and 20% energy to subsequent zones.

In an optimization phase, the initial zone's conic constant is optimized, and the conic constant is maintained across zones. Spherical aberration is balanced across zones based on the aperture size. In some instances, zone power profiles are defined, with 75% of the area configured as the base region, and the myopic region defined as approximately 25% of the area, with non-sequential radius relationships defined. The lens design may then be tested for use across corneal spherical aberrations (−0.1 μm to 0.30 μm), and MTF is tested for maintenance above 0.30 units. Performance improvement with increasing aberration is confirmed. Finally, a zone profile integration process may be used by implementing overlapping power profiles and configuring alternating wavefront aberration signs.

In accordance with the present disclosure, specific MFT thresholds may be accomplished (e.g., ≥0.50 at 50 lp/mm and ≥0.3 at 100 lp/mm) and depth of focus specifications met (>1.0D across various corneal profiles). This is accomplished through a continuous range of focus characteristics while maintaining minimal blur levels.

### EXAMPLES

In accordance with the present disclosure, the above principles are implemented in accordance with the below examples.

In Example 1, an intraocular lens comprises an optic with an anterior surface and a posterior surface arranged about an optical axis. At least one of these surfaces possesses a toric shape and includes a combination of zones that yield alternating signs for a wavefront aberration component, defined using annular terms. This configuration balances focus and maintains modulation transfer function (MTF) values at various spatial frequencies.

In Example 2, the intraocular lens described in Example 1 is a monofocal intraocular lens. It achieves an MTF of at least 0.50 at 50 lp/mm and at least 0.3 at 100 lp/mm.

In Example 3, the intraocular lens of Example 2 is provided, in which different area ratios of the combination of zones are employed. This arrangement allows lenses to provide a continuous range of focus with minimal blur levels.

In Example 4, the intraocular lens of Example 3 is provided, in which aberrations present in different zones of the combined zones ensure through-focus MTF at different frequencies, remaining higher than 0.3 MTF units. This design delivers functional vision with a depth of focus exceeding 1.0D across corneas with aspheric values ranging from 0.0 μm to 0.3 μm.

In Example 5, the intraocular lens of any of the preceding examples further includes annular zones that are power-dependent. These annular regions are divided into a base region and a myopic region, with the base region differing from the myopic region in terms of depth of focus.

In Example 6, the intraocular lens of Example 5 is provided, in which higher-order aberrations are optimized to match any intended corneal spherical aberrations. As a result, the depth of focus remains consistent for at least 0.25 μm on both the positive and negative sides of the spherical component of the cornea for which the monofocal intraocular lens is designed.

In Example 7, the intraocular lens of Example 5 is provided, and has a spherical component in each annular zone optimized for a mid-range cornea. This optimization balances spherical aberration for a 4.5 mm aperture, ensuring the MTF at 50 lp/mm stays above 0.30 MTF units across any human cornea with spherical aberration ranging from −0.1 μm to 0.30 μm, covering all ISO corneas.

In Example 8, the intraocular lens of any of the preceding examples further includes regions with alternating power, extending up to any predefined radius or the lens's physical extent, thereby enabling depth of focus across a range of pupil sizes.

In Example 9, the intraocular lens of any of the preceding examples further includes power profiles of adjacent regions that are continuous across xD. They overlap with the next set of zones, ensuring that MTF at every focal plane remains continuous and maintains values that provide functional vision over a range of foci.

In Example 10, the intraocular lens of any of the preceding examples further includes aberration regions on the anterior or posterior surface can either add or subtract myopic region values from the base power to provide a range of focus. The lens is designed to be insensitive to corneal aberrations, as optimized zones compensate for corneal aberrations over the designed pupil size, regardless of phase introduced in the wavefront entering the lens.

In Example 11, the intraocular lens of any of the preceding examples is a monofocal intraocular lens. Its inner regions have a longer focal length than the outer regions, facilitating vision at myopic distances. These regions alternate in location of focus and optimized aberration pattern, resulting in a through-focus MTF profile that is insensitive to the aberration profile of the incoming wavefront.

In Example 12, an intraocular lens comprises a refractive lens with a surface featuring multiple zones. These zones have a minimum of one refractive power and a maximum of the next refractive power, merged such that an aspheric component minimally impacts visual performance at any radii. The refractive profile is a combination of two or more refractive profiles, each maintaining MTF values without voids in vision.

In Example 13, the intraocular lens of Example 12 is insensitive to corneal aberrations, as the zones compensate for corneal aberrations over designed pupil sizes, regardless of phase introduced in the wavefront entering the monofocal intraocular lens.

In Example 14, a set of intraocular lenses, including refractive zones, comprises a plurality of lenses. Each lens has a different refractive base dioptric power, with zone profiles selected to have a phase delay relative to aqueous fluid at 546 nm light. This phase delay, combined with an overlap method of combining profiles, spreads the light along the depth of focus without affecting the aspheric component over the full optic.

In Example 15, the set of intraocular lenses described in Example 14 is provided in which each lens has a range of radii with an aberration component individually optimized for each radius. This results in optimized aberration for the entire optic and reduced sensitivity to a range of human corneal aberrations.

In Example 16, an ophthalmic lens, such as an intraocular lens, comprises an optic with an anterior surface and a posterior surface about an optical axis. At least one of the surfaces has a profile characterized by the superposition of a base profile and one or more auxiliary profiles. The auxiliary profile is positioned within a boundary, serving as a transition region where the optical path difference corresponds to any arbitrary radius for a design wavelength and includes an aspheric component. This transition region extends from a defined inner radial boundary to an outer radial boundary.

In Example 17, the intraocular lens of Example 16 is provided, in which the auxiliary profile comprises either an auxiliary aspheric profile or an auxiliary non-aspheric profile. The transition region can be adapted to provide a monotonic change in optical path difference relative to its inner radial boundary, based on MTF at a specified spatial frequency, ensuring visual performance.

In Example 18, the intraocular lens of Example 16 is provided, and features a monotonic change in optical path difference, characterized by a continuous increase or decrease as a function of radial distance changes in surface radius.

In Example 19, the intraocular lens of Example 18 is provided, in which changes in surface radius are characterized by either a linear or non-linear change or a succession of radius changes. These changes, with an aspheric component optimized for each zone and a combination of zones, provide optimal aberrations for the entire range of pupil sizes, covering photopic to mesopic light levels.

In Example 20, an intraocular lens comprises an optic with an anterior surface and a posterior surface arranged about an optical axis. At least one of these surfaces has a toric shape, and one surface includes a plurality of concentrically positioned zones. Adjacent zones have alternating signs for a wavefront aberration component, defined using annular terms to balance focus. The zones are selected to maintain MTF values, providing functional vision over a range of focal distances.

In Example 21, the intraocular lens of Example 20 is provided, and includes at least one of a refractive or diffractive lens. The lens features a first zone with a first radius, a second zone with a second radius, and a third zone with a third radius. These radii do not follow a strictly decreasing or increasing progression.

In Example 22, the intraocular lens of Example 21 is provided, in which the zones are sized based on desired energy distribution percentages at different focal distances. Zone areas are calculated using πr² for an internal zone and π(R2²-R1²) for subsequent zones, where R2 represents the outer radius and R1 the inner radius of a given zone.

In Example 23, the intraocular lens of Example 20 is provided, in which a central zone occupies approximately 75% of the area for base power to myopic power, while peripheral zones occupy approximately 25% of the area from mid-point power to 0.5D myopia.

In Example 24, the intraocular lens of Example 21 is provided, and features a base region with a first power and a myopic region with a second power. The base region differs from the myopic region by a designed depth of focus.

In Example 25, the intraocular lens of Example 21 is provided, in which adjacent zones have overlapping power profiles, maintaining continuous MTF values across focal planes.

In Example 26, the intraocular lens of Example 21 is provided, in which each zone includes an optimized conic constant which remains consistent across subsequent zones.

In Example 27, the intraocular lens of Example 21 has power distributions providing 40% of energy directed to distance vision and 20% of energy from subsequent zones.

In Example 28, the intraocular lens of Example 21 is provided and extends to a radius of approximately 1.5 millimeters to accommodate an average three-millimeter diameter pupil.

In further examples, methods of designing and/or optimizing an intraocular lens are described. One method of manufacturing an intraocular lens includes forming an optic having an anterior surface and a posterior surface about an optical axis; forming a plurality of zones on at least one of the anterior surface and the posterior surface by: calculating a first zone area using πr² for an internal zone; calculating subsequent zone areas using π(R2²−R1²), where R2 represents an outer radius and R1 represents an inner radius of each subsequent zone; and sizing the zones based on desired energy distribution percentages at different focal distances. The zones maintain optical performance across different corneal profiles.

Another method of optimizing an intraocular lens includes defining a plurality of concentric zones on at least one surface of an optic, and establishing a base power for a central zone. The method includes establishing a myopic power region extending from the base power, and optimizing a conic constant for an initial zone. The method includes maintaining the optimized conic constant across subsequent zones; and configuring the zones to maintain MTF values above 0.30 units across corneal spherical aberrations ranging from −0.1 to 0.30 micrometers.

A method of designing an intraocular lens includes forming a plurality of zones on at least one surface of an optic; configuring a first set of zones to occupy approximately 75% of area for base power to myopic power; configuring a second set of zones to occupy approximately 25% of area from mid-point power to 0.5D myopia; establishing non-sequential radius relationships between adjacent zones; and optimizing spherical components for mid-range corneal performance.

A method of manufacturing an intraocular lens includes forming an optic with anterior and posterior surfaces; defining multiple zones extending to approximately 1.5 millimeters radius; distributing optical power across the zones by directing approximately 40% of energy to distance vision through a central zone, and directing approximately 20% of energy through subsequent zones; and configuring adjacent zones with overlapping power profiles to maintain continuous MTF values.

A further method of optimizing an intraocular lens includes forming a plurality of zones on at least one surface of an optic; establishing alternating signs for wavefront aberration components between adjacent zones using annular terms; optimizing spherical aberration components within each zone; and configuring power profiles between adjacent zones to overlap for maintaining continuous MTF values across focal planes while preserving corneal independence.

Various embodiments of systems, devices, and methods have been described herein. These embodiments are given only by way of example and are not intended to limit the scope of the claimed subject matter. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed subject matter.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. § 112 (f) are not to be invoked unless the specific terms “means for” or “step for” are recited in a claim.

What is claimed is:

1. An intraocular lens, comprising:
an optic having an anterior surface and a posterior surface disposed about an optical axis, at least one of the anterior surface and the posterior surface having a toric shape and at least one of the anterior surface and the posterior surface including:
a combination of zones in that surface with results having alternating signs for a wavefront aberration component defined using annular terms for balancing focus and maintaining MTF values at different spatial frequencies.

2. The intraocular lens of claim 1, wherein the lens is a monofocal intraocular lens and an MTF at 50 lp/mm and 100 lp/mm is at least 0.50 and at least 0.3 units respectively.

3. The intraocular lens of claim 2, wherein different area ratios of the combination of zones are used such that lenses have a continuous range of focus with very low blur levels.

4. The intraocular lens of claim 3, wherein aberration in different zones of the combination of zones provides through focus MTF at different frequencies that stay higher than 0.3MTF units providing functional vision at a depth of focus greater than 1.0D across corneas of all aspheric values from 0.0 μm to 0.3 μm.

5. The intraocular lens of claim 1, further comprising annular zones which are power dependent such that annular regions are split into base region and myopic region with the base region differing from myopic region by depth of focus.

6. The intraocular lens of claim 5, wherein higher order aberrations are optimized to any intended corneal spherical aberrations such that the depth of focus is consistent for at least 0.25 μm on both positive and negative sides of a spherical component of a cornea for which the monofocal intraocular lens is designed.

7. The intraocular lens of claim 5, wherein a spherical component of each annular zone is optimized for a mid-range cornea such that a spherical aberration is balanced for a 4.5 mm aperture such that MTF at 50 lp/mm stays above 0.30 MTF units across any human cornea with the spherical aberration in the range −0.1 μm and 0.30 μm which includes all ISO corneas.

8. The intraocular lens of claim 1, wherein regions of alternating power can extend up to any predefined radius or to the physical extent of the lens itself to enable depth of focus over a range of pupil sizes.

9. The intraocular lens of claim 1, wherein power profiles of adjacent regions are continuous over xD and overlap with a next set of zones such that MTF at every focal plane is continuous and is maintained at values providing functional vision over a range of foci.

10. The intraocular lens of claim 1, wherein:
aberration regions on the anterior surface or the posterior surface can either add or subtract myopic region values from base power to provide a range of focus; and
the intraocular lens is insensitive to corneal aberrations as optimized zones provide compensation for corneal aberrations over designed pupil size irrespective of phase introduced in a wavefront entering the intraocular lens.

11. The intraocular lens of claim 1, wherein the intraocular lens comprises a monofocal intraocular lens, and wherein inner regions have a longer focal length than outer regions facilitating vision at myopic distances and wherein these regions are alternating a location of focus and optimized aberration pattern resulting in a through focus MTF profile which is insensitive to the aberration profile of the incoming wavefront.

12. An intraocular lens, comprising:
a refractive lens with a surface having multiple zones on the surface with a minimum of one refractive power and a maximum of a next refractive power merged such that an aspheric component minimally impacts visual performance of the intraocular lens at any radii;
wherein a refractive profile is comprised of a combination of two or more refractive profiles, each having a power so as to maintain MTF values without voids in vision.

13. The intraocular lens of claim 12, wherein the intraocular lens is insensitive to corneal aberrations as the zones provide compensation for corneal aberrations over designed pupil sizes irrespective of phase introduced in a wavefront entering the monofocal intraocular lens.

14. A set of intraocular lenses including refractive zones, comprising:
a plurality of lenses, each of the lenses having a different refractive, base dioptric power than one another, the plurality of lenses having zone profiles;
wherein the zone profiles are selected to have a phase delay, relative to aqueous fluid with any range of power designed at 546 nm light, which in conjunction with an overlap method of combining profiles tends to spread the light along the depth of focus without affecting an aspheric component over the full optic.

15. The set of intraocular lenses of claim 14, wherein the plurality of lenses each has a range of radii with an aberration component individually optimized for each radius resulting in optimized aberration for an entire optic and reduced sensitivity to a range of human corneal aberrations.

16. An ophthalmic lens, such as an intraocular lens, comprising:
an optic having an anterior surface and a posterior surface about an optical axis, wherein at least one of the surfaces has a profile characterized by superposition of a base profile and one or more auxiliary profile;
wherein the auxiliary profile is positioned within a boundary and serves as a transition region, wherein an optical path difference across the transition region corresponds to any arbitrary radius for a design wavelength and includes an aspheric component;
wherein the transition region of the auxiliary profile extends from a defined inner radial boundary to an outer radial boundary.

17. The intraocular lens of claim 16, wherein the auxiliary profile comprises an auxiliary aspheric profile or an auxiliary non-aspheric profile, and wherein the transition region can be adapted to provide a monotonic change in optical path difference relative to its inner radial boundary as a function of MTF at a specified spatial frequency to provide visual performance.

18. The intraocular lens of claim 16, wherein the monotonic change in optical path difference is characterized by a continuous increase or decrease as a function of radial distance of changes in surface radius.

19. The intraocular lens of claim 18, wherein changes in surface radius are characterized by at least one of (1) a linear or a non-linear change or (2) a succession of radius changes with an aspheric component optimized for each zone of a plurality of zones and a combination of zones to provide optimal aberrations for an entire range of pupil sizes covering photopic to mesopic light levels.

20. An intraocular lens, comprising:
an optic having an anterior surface and a posterior surface disposed about an optical axis, at least one of the anterior surface and the posterior surface having a toric shape and a shaped surface selected from the anterior surface and the posterior surface,
wherein the shaped surface includes a plurality of zones positioned concentrically, and
wherein adjacent ones of the plurality of zones having alternating signs for a wavefront aberration component defined using annular terms for balancing focus, the plurality of zones being selecting to maintain modulation transfer function (MTF) values providing functional vision over a range of focal distances.

21. The intraocular lens of claim 20, wherein the intraocular lens comprises at least one of a refractive lens or a diffractive lens, and wherein the plurality of zones includes:
a first zone having a first radius;
a second zone having a second radius; and
a third zone having a third radius;
wherein the first, second, and third radii have a non-sequential relationship such that they are not required to follow a strictly decreasing or increasing progression.

22. The intraocular lens of claim 21, wherein the plurality of zones are sized according to desired energy distribution percentages at different focal distances, with zone areas calculated using:
πr² for an internal zone; and
π(R2²-R1²) for subsequent zones;
wherein R2 represents an outer radius and R1 represents an inner radius of a given zone.

23. The intraocular lens of claim 21, wherein:
a central zone occupies approximately 75% of area for base power to myopic power; and
peripheral zones occupy approximately 25% of area from mid-point power to 0.5D myopia.

24. The intraocular lens of claim 21, wherein the plurality of zones comprises:
a base region having a first power; and
a myopic region having a second power;
wherein the base region differs from the myopic region by a designed depth of focus.

25. The intraocular lens of claim 21, wherein adjacent zones have overlapping power profiles configured to maintain continuous MTF values across focal planes.

26. The intraocular lens of claim 21, wherein:
each zone includes an optimized conic constant; and
the optimized conic constant is maintained consistent across subsequent zones.

27. The intraocular lens of claim 21, wherein the plurality of zones are configured with power distributions that provide:
40% of energy directed to distance vision; and
20% of energy directed from subsequent zones.

28. The intraocular lens of claim 21, wherein the zones extend to a radius of approximately 1.5 millimeters to accommodate an average three millimeter diameter pupil.
